(19) United States
(12) Patent Application Publication
(10) Pub. No.: US 2026/0367092 A1
(43) Pub. Date: Nov. 19, 2026

(54) HELICOBACTER PYLORI DETECTION METHOD BASED ON MASS SPECTROMETRY IDENTIFICATION

(71) Applicant: LANZHOU BAIYUAN GENE TECHNOLOGY CO., LTD., Lanzhou (CN)

(72) Inventor: Tuanjie CHE, Lanzhou (CN)

(73) Assignee: LANZHOU BAIYUAN GENE TECHNOLOGY CO., LTD., Lanzhou (CN)

(21) Appl. No.: 19/340,896

(22) Filed: Sep. 26, 2025

Related U.S. Application Data
(63) Continuation of application No. PCT/CN2025/113405, filed on Aug. 8, 2025.

(30) Foreign Application Priority Data
May 15, 2025 (CN) 202510623071.5

(51) Int. Cl. G01N 33/68; G16B 40/20; H01J 49/00

(52) U.S. Cl. CPC G01N 33/6851; G16B 40/20; H01J 49/0036; G01N 2333/195

(57) ABSTRACT

A Helicobacter pylori detection method based on mass spectrometry identification first acquires MALDI mass spectra of the sample under test to obtain its protein fingerprint spectrum, which is then preprocessed to remove noise, followed by extraction of visual pattern features of the feature under test with discriminative ability. Reference visual pattern features of the known bacterial species are extracted from a previously constructed reference database. A complex matching relationship between the feature under test and the reference feature is identified by introducing protein fingerprint spectrum pattern feature query-response analysis based on a feature domain to obtain a query-response encoding representation of the fingerprint spectrum visual pattern feature, and a final bacterial species detection result is determined based on the query response. The method is intended to fully exploit deep pattern information embedded in mass spectrometry data, thereby enabling more reliable mass spectrometry identification of H. pylori.

### CROSS REFERENCE TO THE RELATED APPLICATIONS

This application is a continuation application of International Application No. PCT/CN2025/113405, filed on Aug. 8, 2025, which is based upon and claims priority to Chinese Patent Application No. 202510623071.5, filed on May 15, 2025, the entire contents of which are incorporated herein by reference.

### TECHNICAL FIELD

The present disclosure relates to the technical field of Helicobacter pylori detection, and in particular to a Helicobacter pylori detection method based on mass spectrometry identification.

### BACKGROUND

Helicobacter pylori (H. pylori) is a common Gram-negative bacterium that primarily colonizes the human gastric mucosa. It is a major pathogenic factor of various upper gastrointestinal diseases, including chronic gastritis, peptic ulcers, gastric mucosa-associated lymphoid tissue lymphoma, and even gastric cancer. Given its high infection rate and serious health consequences, the development of rapid, accurate, and reliable detection methods for H. pylori is of critical clinical significance and public health value for early diagnosis, effective treatment, and the prevention of severe complications such as gastric cancer.

In recent years, matrix-assisted laser desorption/ionization time-of-flight mass spectrometry (MALDI-TOF MS) has been widely applied in microbial identification due to its rapidity, high throughput, and relatively low cost. Existing mass spectrometry-based microbial detection methods typically acquire protein fingerprints of the target microorganisms (either through culture or direct enrichment from samples) and then identify the species by comparing the resulting mass spectral peak list or the full spectrum with reference spectra of known strains stored in a database. However, when applied to the detection of specific bacterial species such as H. pylori, these traditional calculation methods based on peak matching or overall spectrum similarity have some limitations. For example, subtle differences in protein expression among different H. pylori strains or between H. pylori and other commensal or pathogenic bacteria may result in mass spectra that are overall similar but differ in key characteristic peaks. In addition, factors such as sample preparation, culture conditions, and instrument status can cause spectral drift and fluctuations in peak intensity. These changes may interfere with the accuracy of conventional comparison algorithms, especially when processing low-quality spectra or distinguishing closely related bacterial strains, and may easily lead to misidentification or identification failure, thereby reducing both sensitivity and specificity.

Therefore, there is a need for an optimized H. pylori detection method based on mass spectrometry identification.

### SUMMARY

In order to solve the above technical problems, the present disclosure provides a Helicobacter pylori detection method based on mass spectrometry identification. The Helicobacter pylori detection method based on mass spectrometry identification provided by the present disclosure adopts a strategy of feature extraction and query network matching based on the visual pattern features, which is intended to fully exploit deep pattern information embedded in mass spectrometry data, thereby enabling more reliable mass spectrometry identification of Helicobacter pylori (H. pylori).

According to one aspect of the present disclosure, a Helicobacter pylori detection method based on mass spectrometry identification is provided. The method includes: mixing a sample under test with a matrix-assisted laser desorption/ionization (MALDI) matrix to obtain a mixture, spotting the mixture onto a MALDI target plate, and acquiring a protein fingerprint spectrum using a MALDI-TOF mass spectrometer; extracting a set of protein reference fingerprint spectra of a known bacterial species from a bacterial species reference database; preprocessing the protein fingerprint spectrum to obtain a preprocessed protein fingerprint spectrum; extracting a fingerprint spectrum visual pattern feature from the preprocessed protein fingerprint spectrum to obtain a visual pattern feature of the protein fingerprint spectrum under test; extracting fingerprint spectrum visual pattern features from the set of protein reference fingerprint spectra of the known bacterial species to obtain a set of protein fingerprint spectrum reference visual pattern features; performing a fingerprint spectrum visual pattern feature query on the protein fingerprint spectrum under test and the set of protein fingerprint spectrum reference visual pattern features to obtain a response feature for the fingerprint spectrum visual pattern feature query; and determining a bacterial species detection result based on the response feature for the fingerprint spectrum visual pattern feature query.

In one possible implementation mode, the preprocessing includes baseline correction, noise smoothing, peak detection, and alignment.

In one possible implementation mode, the extracting a fingerprint spectrum visual pattern feature from the preprocessed protein fingerprint spectrum to obtain a visual pattern feature of a protein fingerprint spectrum under test includes: processing the preprocessed protein fingerprint spectrum through a fingerprint spectrum visual pattern feature extractor based on a dilated convolutional neural network model to obtain a visual pattern feature encoding vector of the protein fingerprint spectrum under test, which serves as the visual pattern feature of the protein fingerprint spectrum under test.

In one possible implementation mode, the extracting fingerprint spectrum visual pattern features from the set of protein reference fingerprint spectra of the known bacterial species to obtain a set of protein fingerprint spectrum reference visual pattern features includes: processing each protein reference fingerprint spectrum of the known bacterial species in the set of protein reference fingerprint spectra of the known bacterial species through the fingerprint spectrum visual pattern feature extractor based on the dilated convolutional neural network model to obtain a set of protein fingerprint spectrum reference visual pattern feature encoding vectors, which serves as the set of protein fingerprint spectrum reference visual pattern features.

In one possible implementation mode, the performing a fingerprint spectrum visual pattern feature query on the protein fingerprint spectrum under test and the set of protein fingerprint spectrum reference visual pattern features to obtain a response feature for the fingerprint spectrum visual pattern feature query includes: after performing a feature enhancement on the visual pattern feature encoding vector of the protein fingerprint spectrum under test, performing an individual semantic query encoding on the visual pattern feature encoding vector of the protein fingerprint spectrum under test and each protein fingerprint spectrum reference visual pattern feature encoding vector in the set of protein fingerprint spectrum reference visual pattern feature encoding vectors respectively to obtain a set of individual semantic query score encoding vectors for the visual pattern of the protein fingerprint spectrum under test; calculating an individual semantic weight of each individual semantic query score encoding vector for the visual pattern of the protein fingerprint spectrum under test in the set of individual semantic query score encoding vectors for the visual pattern of the protein fingerprint spectrum under test to obtain a set of semantic self-attention weights for individual query of the visual pattern of the protein fingerprint spectrum under test; and aggregating the set of individual semantic query score encoding vectors for the visual pattern of the protein fingerprint spectrum under test based on the set of semantic self-attention weights for individual query of the visual pattern of the protein fingerprint spectrum under test to obtain the fingerprint spectrum visual pattern feature query-response encoding vector.

In one possible implementation mode, the after performing a feature enhancement on the visual pattern feature encoding vector of the protein fingerprint spectrum under test, performing an individual semantic query encoding on the visual pattern feature encoding vector of the protein fingerprint spectrum under test and each protein fingerprint spectrum reference visual pattern feature encoding vector in the set of protein fingerprint spectrum reference visual pattern feature encoding vectors respectively to obtain a set of individual semantic query score encoding vectors for the visual pattern of the protein fingerprint spectrum under test includes: performing a feature enhancement based on deconvolutional encoding on the visual pattern feature encoding vector of the protein fingerprint spectrum under test to obtain an enhanced visual pattern feature encoding vector of the protein fingerprint spectrum under test, where the enhanced visual pattern feature encoding vector of the protein fingerprint spectrum under test has a feature scale same as that of each protein fingerprint spectrum reference visual pattern feature encoding vector in the set of protein fingerprint spectrum reference visual pattern feature encoding vectors; and performing an individual semantic query encoding on the enhanced visual pattern feature encoding vector of the protein fingerprint spectrum under test and each protein fingerprint spectrum reference visual pattern feature encoding vector in the set of protein fingerprint spectrum reference visual pattern feature encoding vectors respectively to obtain the set of individual semantic query score encoding vectors for the visual pattern of the protein fingerprint spectrum under test.

In one possible implementation mode, the calculating an individual semantic weight of each individual semantic query score encoding vector for the visual pattern of the protein fingerprint spectrum under test in the set of individual semantic query score encoding vectors for the visual pattern of the protein fingerprint spectrum under test to obtain a set of semantic self-attention weights for individual query of the visual pattern of the protein fingerprint spectrum under test respectively includes: determining an individual semantic matching degree for each individual semantic query score encoding vector for the visual pattern of the protein fingerprint spectrum under test in the set of individual semantic query score encoding vectors for the visual pattern of the protein fingerprint spectrum under test to obtain a set of individual semantic matching degrees for the visual pattern of the protein fingerprint spectrum under test according to self-distribution characteristics of the set of individual semantic query score encoding vectors for the visual pattern of the protein fingerprint spectrum under test; and inputting the set of individual semantic matching degrees for the visual pattern of the protein fingerprint spectrum under test into a relation-gated agent module to obtain a set of semantic self-attention weights for individual query of the visual pattern of the protein fingerprint spectrum under test.

In one possible implementation mode, the determining an individual semantic matching degree for each individual semantic query score encoding vector for the visual pattern of the protein fingerprint spectrum under test in the set of individual semantic query score encoding vectors for the visual pattern of the protein fingerprint spectrum under test to obtain a set of individual semantic matching degrees for the visual pattern of the protein fingerprint spectrum under test according to self-distribution characteristics of the set of individual semantic query score encoding vectors for the visual pattern of the protein fingerprint spectrum under test includes: performing intrinsic alignment calibration on each individual semantic query score encoding vector for the visual pattern of the protein fingerprint spectrum under test in the set of individual semantic query score encoding vectors for the visual pattern of the protein fingerprint spectrum under test to obtain a set of calibrated individual semantic query score encoding vectors for the visual pattern of the protein fingerprint spectrum under test; and calculating an individual semantic matching degree for each calibrated individual semantic query score encoding vector for the visual pattern of the protein fingerprint spectrum under test respectively according to distribution characteristics of the set of calibrated individual semantic query score encoding vectors for the visual pattern of the protein fingerprint spectrum under test to obtain the set of individual semantic matching degrees for the visual pattern of the protein fingerprint spectrum under test.

In one possible implementation mode, the determining a bacterial species detection result based on the response feature for the fingerprint spectrum visual pattern feature query includes: processing the fingerprint spectrum visual pattern feature query-response encoding vector through a classifier-based bacterial species detector to obtain the bacterial species detection result, where the bacterial species detection result is used to represent a bacterial species category label.

The present disclosure provides a Helicobacter pylori detection method based on mass spectrometry identification, which first acquires MALDI mass spectra of the sample under test to obtain its protein fingerprint spectrum. The spectrum is then preprocessed to remove noise and is standardized, followed by extraction of visual pattern features of a feature under test with discriminative ability. Reference visual pattern features of the known bacterial species are extracted from a previously constructed reference database. Finally, a complex matching relationship between the feature under test and a reference feature is identified by introducing protein fingerprint spectrum pattern feature query-response analysis based on a feature domain to obtain a query-response encoding representation of the fingerprint spectrum visual pattern feature, and a final bacterial species detection result is determined based on the query response. This strategy of feature extraction and query network matching based on the visual pattern features is intended to fully exploit deep pattern information embedded in mass spectrometry data, thereby enabling more reliable mass spectrometry identification of H. pylori.

### BRIEF DESCRIPTION OF THE DRAWINGS

In order to more clearly illustrate the technical solutions in the embodiments of the present disclosure, the accompanying drawings of the embodiments will be briefly described below. Obviously, the accompanying drawings described below merely illustrate some embodiments of the present disclosure and are not intended to limit the present disclosure.

FIG. 1 is a schematic flow chart of a Helicobacter pylori detection method based on mass spectrometry identification according to an embodiment of the present disclosure.

FIG. 2 is a schematic diagram of data flow of a Helicobacter pylori detection method based on mass spectrometry identification according to an embodiment of the present disclosure.

FIG. 3 is a schematic flow chart of a step S6 of a Helicobacter pylori detection method based on mass spectrometry identification according to an embodiment of the present disclosure.

FIG. 4 is a schematic flow chart of a step S61 of a Helicobacter pylori detection method based on mass spectrometry identification according to an embodiment of the present disclosure.

FIG. 5 is a schematic flow chart of a step S62 of a Helicobacter pylori detection method based on mass spectrometry identification according to an embodiment of the present disclosure.

### DETAILED DESCRIPTION OF THE EMBODIMENTS

The technical solutions of embodiments of the present disclosure will be described below clearly and comprehensively in conjunction with accompanying drawings of the embodiments of the present disclosure. Apparently, the embodiments described are merely some embodiments rather than all embodiments of the present disclosure. On the basis of the embodiments of the present disclosure, all other embodiments obtained by those of ordinary skill in the art without making creative efforts shall fall within the scope of protection of the present disclosure.

In view of the foregoing technical problems, the present disclosure provides a Helicobacter pylori detection method based on mass spectrometry identification, aiming to improve the accuracy and robustness of mass spectrometry-based detection of Helicobacter pylori (H. pylori). A key aspect of the method lies in no longer relying solely on discrete peak positions and peak intensities, but rather in focusing on extracting a fingerprint spectrum visual pattern feature from protein fingerprint spectra capable of better reflecting overall spectral morphology and structural information after preprocessing procedures such as baseline correction, noise smoothing, peak identification, and alignment. That is, the protein fingerprint spectra, similar to images, contain abundant pattern information. Extracting a visual pattern feature from protein fingerprint spectra is capable of capturing macroscopic structure and characteristic distribution of key regions in the protein fingerprint spectra. The feature may be more resistant to interference from noise and subtle variations than the mere presence or precise location of individual peaks, thereby representing an identity of a bacterial species in a more stable manner. Correspondingly, reference protein fingerprint spectra of a known bacterial species in a bacterial species reference database are also subjected to the same process to extract reference visual pattern features, thereby constructing a reference database based on the visual pattern features. Further, to achieve efficient and accurate matching, this solution introduces protein fingerprint spectrum pattern feature query-response analysis based on a feature domain to learn a complex matching relationship between a feature under test and a reference feature. A query dilated convolutional neural network is configured to go beyond simple distance calculations or similarity scoring, and to intelligently determine which reference bacterial species exhibits the most similar visual pattern to that of a sample under test. In doing so, the query network outputs a query-response encoding representation of the fingerprint spectrum visual pattern feature, and a final bacterial species detection result is determined based on the query response. This strategy of feature extraction and query network matching based on the visual pattern features is intended to fully exploit deep pattern information embedded in mass spectrometry data, thereby enabling more reliable mass spectrometry identification of H. pylori.

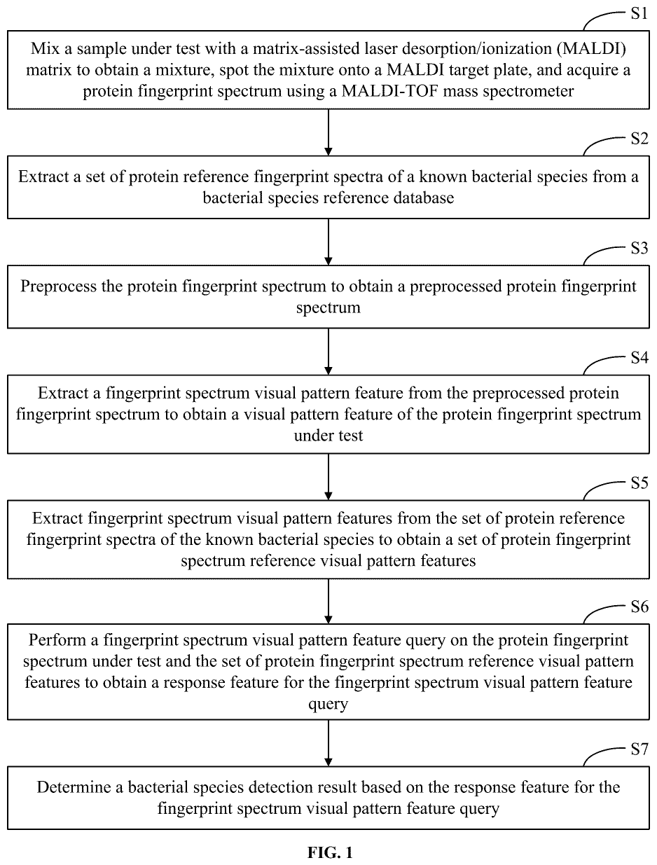

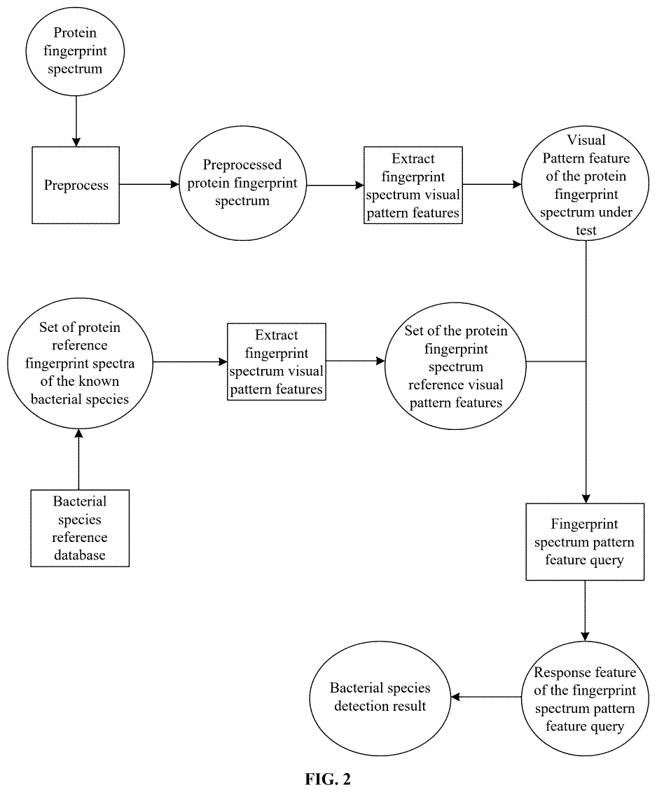

To address the above technical problems, the present disclosure provides a Helicobacter pylori detection method based on mass spectrometry identification. FIG. 1 illustrates a schematic flowchart of a Helicobacter pylori detection method based on mass spectrometry identification according to an embodiment of the present disclosure. FIG. 2 illustrates a schematic diagram of data flow of a Helicobacter pylori detection method based on mass spectrometry identification. As shown in FIGS. 1 and 2, the Helicobacter pylori detection method based on mass spectrometry identification includes the following steps: S1: mixing a sample under test with a matrix-assisted laser desorption/ionization (MALDI) matrix to obtain a mixture, spotting the mixture onto a MALDI target plate, and acquiring a protein fingerprint spectrum using a MALDI-TOF mass spectrometer; S2: extracting a set of protein reference fingerprint spectra of a known bacterial species from a bacterial species reference database; S3: preprocessing the protein fingerprint spectrum to obtain a preprocessed protein fingerprint spectrum; S4: extracting a fingerprint spectrum visual pattern feature from the preprocessed protein fingerprint spectrum to obtain a visual pattern feature of a protein fingerprint spectrum under test; S5: extracting fingerprint spectrum visual pattern features from the set of protein reference fingerprint spectra of the known bacterial species to obtain a set of protein fingerprint spectrum reference visual pattern features; S6: performing a fingerprint spectrum visual pattern feature query on the protein fingerprint spectrum under test and the set of protein fingerprint spectrum reference visual pattern features to obtain a response feature for the fingerprint spectrum visual pattern feature query; and S7: determining a bacterial species detection result based on the response feature for the fingerprint spectrum visual pattern feature query.

Specifically, in the step S1, a sample under test is mixed with a MALDI matrix to obtain a mixture, the mixture is spotted onto a MALDI target plate, and a protein fingerprint spectrum is acquired using a MALDI-TOF mass spectrometer. It should be understood that, in a proteomics analysis, the MALDI matrix (such as α-cyano-4-hydroxycinnamic acid) may facilitate effective ionization of peptides or proteins in the sample, essentially suppressing interference and enhancing signal intensities. A biological sample under test (such as wet cultured H. pylori cells) is suspended in a suitable solution at a specified concentration, and then thoroughly mixed with a MALDI matrix solution prepared in a defined ratio to ensure adequate contact between the proteins and matrix crystals. Subsequently, an appropriate amount of the mixture (such as 1 μL) is dispensed into designated wells of the MALDI target plate and air-dried at room temperature, thereby enabling proteins/peptides to be evenly embedded within the matrix crystals. This solid-phase spotting method significantly enhances a signal-to-noise ratio and spectral reproducibility of subsequent analysis. After sample preparation is completed, the MALDI target plate is loaded into the MALDI-TOF mass spectrometer. According to preset instrument parameters (such as laser energy and pulse frequency), the sample is scanned point by point using the MALDI-TOF mass spectrometer. Laser pulses irradiate sample-matrix mixture spots. Matrix molecules absorb excitation energy to desorb and ionize the proteins/peptides embedded therein, thereby obtaining time-of-flight distribution information of charged ions. The resulting spectra are internally calibrated by an instrument and recorded by an external data acquisition system, and a fingerprint mass spectrum for each sample under test is finally output, typically represented as a two-dimensional spectrum of a mass-to-charge ratio (m/z) and intensity. For example, H. pylori cells cultured under specific conditions are collected by centrifugation, diluted, and then mixed with a MALDI matrix solution at a ratio of 1:1. 1 μL of the mixture is pipetted and precisely dispensed into designated wells of the MALDI target plate, air-dried to form a uniform thin film, and loaded onto the target plate to acquire protein fingerprint spectra step by step according to preset target points.

Specifically, in the step S2, a set of protein reference fingerprint spectra of a known bacterial species is extracted from a bacterial species reference database. It should be understood that, in clinical or practical microbial detection scenarios, H. pylori not only exhibits diverse strain profiles but is also highly susceptible to interference from various factors such as sample processing, culture conditions, and experimental instruments. These factors may result in spectral drift, fluctuations in peak intensity, or even disappearance or emergence of characteristic peaks across different batches or different laboratories. Conventional comparison algorithms based on peak values or simple full-spectrum similarity, which rely on discrete peak table information and superficial feature distribution, are hard to address the potential complex variations and subtle differences inherent in protein spectra between H. pylori and closely related species. Therefore, in the present disclosure, a set of protein fingerprint spectra of the known bacterial species (including standard bacterial species of H. pylori at various levels) are standardized and incorporated into the bacterial species reference database.

Specifically, in the step S3, the protein fingerprint spectrum is preprocessed to obtain a preprocessed protein fingerprint spectrum. It should be understood that raw MALDI-TOF mass spectrometry data are often subject to interference from various factors such as sample preparation, culture conditions, and instrument status, which may cause baseline drift, high levels of random noise, fluctuations in peak intensity, and slight shifts in peak positions (that is, spectral drift). These imperfections in raw data significantly interfere with subsequent analysis. Both conventional peak-matching methods and feature-based visual pattern methods require relatively clean and standardized data inputs. Specifically, in a technical solution of the present disclosure, the preprocessing includes baseline correction, noise smoothing, peak detection, and alignment. Baseline correction aims to eliminate slowly varying background signals arising from the matrix or other non-peptide substances, thereby ensuring that peak heights can accurately reflect peptide abundance. Noise smoothing aims to reduce high-frequency random fluctuations in the spectrum, and improve the signal-to-noise ratio, thereby making true protein peaks easily detected. Peak detection (or peak extraction) aims to accurately identify characteristic peaks representing proteins or peptide fragments from a smoothed spectrum, determining their m/z positions and intensity values. Alignment aims to correct systematic peak position shifts across different samples or repeated measurements of the same sample, caused by subtle instrument differences or calibration issues, ensuring that a same biomarker appears at comparable positions across different spectra.

Specifically, baseline correction is a first step in mass spectrometry data processing, with a fundamental objective of removing background interference signals in the spectrum. The background signals mainly originate from matrix residue, ion suppression effect, and accumulation of nonspecific ions during analysis, resulting in a “pseudo-baseline” phenomenon represented by upward shifts or slow undulations in a low-frequency region of the spectrum. Algorithms commonly used for baseline correction include a rolling ball algorithm, a Top-Hat filtering algorithm, a least-squares fitting algorithm, and the like. The rolling ball algorithm simulates a lower boundary of a background curve by sliding a ball of a defined window size along a time-of-flight axis, thereby removing the baseline. For example, a window range around an m/z line is set to 200 Da, a baseline is fitted using a lower edge of a simulated rolling ball closely following the line to obtain local minimum points, and a baseline profile is subtracted point by point from a raw signal to restore true peak heights. The Top-Hat filtering algorithm suppresses slow-varying components of the spectrum by constructing a morphological structural element. The least-squares fitting algorithm employs a polynomial fit to form a background parabola, and a correction is achieved by subtracting a background function from an original spectrum. High-quality baseline correction ensures that signal intensity reflects only true protein peak concentrations, effectively suppressing interference from false signals.

After baseline correction is completed, noise smoothing needs to be performed on the remaining signal. High-frequency noise in the mass spectrometry data mainly manifests as random spikes, noise bands, or subtle periodic fluctuations. Effective noise smoothing retains sharpness and contrast of true peak shapes while eliminating non-biologically relevant signal fluctuations. Commonly used noise smoothing methods include a moving average method, a Gaussian filtering method, a Savitzky-Golay smoothing method, and the like. The moving average method computes an average by sliding a fixed window (such as 5-11 points), which is effective for reducing high-frequency noise, but may slightly reduce resolution. The Gaussian filtering method performs a weighted average of raw signals using a Gaussian kernel, thereby filtering out abrupt noise while preserving the fidelity of the peak shape. The Savitzky-Golay smoothing method fits the signal within a local sliding window using a low-order polynomial, balancing noise suppression with preservation of peak shape details. Taking MALDI-TOF proteomic data as an example, a window width is typically set to 7-15 points, a polynomial order is set to 2-3, and parameters may be adjusted according to a balance between the specific spectrum quality and the desired resolution. Through multiple experiments and empirical evaluations, a standardized signal trajectory with a distinct peak structure, a low-noise background, and a naturally smooth peak shape is finally obtained.

Subsequently, based on the noise-smoothed spectrum, peak identification processing is performed with an objective of accurately locating protein/polypeptide characteristic peaks within the spectrum and extracting key informational parameters thereof (such as m/z value, peak height, peak area, and peak width). The peak identification process involves dynamic and adaptive adjustments according to spectral characteristics such as overall signal-to-noise ratio, local noise level, and inter-peak spacing. Commonly used methods include a threshold determination based on local maxima, a peak shape determination method using first-order and second-order derivatives, and a multi-scale detection method incorporating prior biological information. In practical operation, spectral data points are traversed in a sliding-window manner. For each data point, difference values of its adjacent points are calculated. When an intensity at a point is greater than those of its nearest left and right neighboring data points and reaches a preset signal-to-noise threshold (for example, S/N>3), and its locally fitted peak width and peak area conform to the protein peak distribution characteristics, the point is identified as a valid peak position. For complex samples (such as H. pylori proteome spectra), breakpoint detection, continuous wave peak analysis, and other signal processing techniques may be introduced to merge weak peaks and false peaks detected from repeated measurements, thereby improving the accuracy of peak identification. Parameters for peak identification, such as minimum peak height, minimum peak width, and minimum peak area, are flexibly set according to the performance of the mass spectrometer and the specific experimental system.

After precise peak identification is completed, cross-batch sample data or repeated measurements of a single sample often exhibit slight shifts of the same protein peak along the x-axis (m/z dimension) due to ion source energy drift, timing adjustment errors, and the like. In order to ensure effective comparison of peak information across the spectra, and between the reference database and the sample under test, peak alignment processing is required for all spectra. Peak alignment methods include a global linear mapping method, a local nonlinear registration method, and a marker-based dynamic time normalization method. The linear mapping method assumes a constant drift throughout the spectrum. By calculating the m/z deviations of internal standard peaks or strong characteristic peaks in different samples, the method performs a linear regression to help the calibration throughout the spectrum. For a complex drift pattern, the nonlinear registration method may be employed. A set of co-occurring peaks (that is, homologous marker peak groups) is first identified in each spectrum, and a dynamic time warping algorithm is adopted to match m/z sequences, thereby achieving global and local non-uniform mapping. In addition, the existing advanced spectrum alignment toolkits provide automated batch alignment of multiple spectra. Interpolation weighting, time-domain normalization, waveform hierarchical clustering, and other techniques may be used at an underlying level, to ensure consistency of the correspondence of each recognized peak across all samples or all database reference spectra.

In one specific embodiment, after a protein fingerprint spectrum is collected, raw profile-type data is first loaded into professional data processing software. The baseline correction is performed using the rolling ball algorithm with a window size of 15 Da, followed by Savitzky-Golay filtering with a width of 7 points for noise reduction. Valid peaks are then identified using a local maximum detection method with a signal-to-noise threshold of 3. Thereafter, 3-5 internal standard peaks are selected to perform linear and/or nonlinear alignment mapping, obtaining a preprocessed protein fingerprint spectrum.

Specifically, in the step S4, fingerprint spectrum visual pattern features are extracted from the preprocessed protein fingerprint spectrum to obtain a fingerprint spectrum visual pattern feature of the protein fingerprint spectrum under test. It should be understood that, due to over-reliance on discrete peak position, peak intensity information, or overall spectral similarity, conventional H. pylori detection methods are susceptible to spectral drift, fluctuations in peak intensity, and subtle differences between the strains caused by sample preparation and instrument conditions, leading to insufficient accuracy and stability of identification. On this basis, in order to go beyond the variable information, and explore extract fingerprint spectrum visual pattern features capable of better reflecting stable intrinsic structure of the spectrum, a technical solution of the present disclosure further extracts a fingerprint spectrum visual pattern feature from the preprocessed protein fingerprint spectrum to obtain a visual pattern feature encoding vector of a protein fingerprint spectrum under test. The fingerprint spectrum visual pattern features, similar to images, contain abundant pattern information about macroscopic structure and key regional distributions, and are more resistant to noise and subtle variations, providing a more stable representation of bacterial species identity. Therefore, by extracting the fingerprint spectrum visual pattern features, features capable of capturing deep structural information, such as overall morphology, peak distribution pattern, and inter-peak relationships, may be extracted from the preprocessed protein fingerprint spectrum.

Specifically, in one embodiment, the extracting a fingerprint spectrum visual pattern feature from the preprocessed protein fingerprint spectrum to obtain a visual pattern feature of the protein fingerprint spectrum under test includes: processing the preprocessed protein fingerprint spectrum through a fingerprint spectrum visual pattern feature extractor based on a dilated convolutional neural network model to obtain a visual pattern feature encoding vector of the protein fingerprint spectrum under test, which serves as a visual pattern feature of the protein fingerprint spectrum under test. Models such as the dilated convolutional neural network model are intended to leverage their capability to effectively enlarge receptive field, capture long-range dependencies, and extract multi-scale features when processing image-like data of the fingerprint spectra, such that complex global and local pattern features in the mass spectrum are captured in a more comprehensive manner, and rather than merely focusing on isolated peaks. An ultimate objective is to transform these complex visual pattern features into a compact and information-rich mathematical representation, that is, the visual pattern feature encoding vector of a protein fingerprint spectrum under test. The encoding vector constitutes a highly generalized and abstracted representation of visual features of the original spectrum, aiming to maximize the preservation of discriminative information while filtering out irrelevant variations.

Specifically, dilated convolution introduces a dilated sampling mechanism, which can significantly enlarge the receptive field, and simultaneously account for both local peak regions and global trends maintaining kernel parameters unchanged. A specific network structure is generally configured as a multi-layer stack, with each layer adopting a different dilation rate to capture multi-scale feature information. In one specific embodiment, the fingerprint spectrum visual pattern feature extractor based on a dilated convolutional neural network model is provided with three convolutional layers in sequence, that is a first convolutional layer (64 channels with a kernel width of 3 and a dilation rate of 1), a second convolutional layer (128 channels with a kernel width of 3 and a dilation rate of 2), and a third convolutional layer (256 channels with a kernel width of 3 and a dilation rate of 4). Batch normalization and ReLU activation are inserted between each layer, and global average pooling is applied as an output layer. Spatial feature vectors of all channels are averaged in a channel direction and compressed into a 256-dimensional visual pattern feature encoding vector of the protein fingerprint spectrum under test. At a training phase, a large number of reference bacterial species fingerprint spectra and class labels from the reference database participate jointly are required. Supervised learning is employed, a cross-entropy loss or triplet loss function is defined to guide categorization and separation of a feature encoding space. Parameter optimization is performed using adaptive gradient descent methods such as Adam, with a learning rate set from 1e-3 to 1e-5, a batch size set from 32 to 128, and a training epoch set from 10 to 100, specifically depending on a training set capacity and a model convergence property. Mechanisms such as Dropout and L2 regularization may be introduced during the model training to prevent overfitting, and data augmentation strategies such as spectral shifting and peak-width perturbation may also be used to improve data diversity. Finally, parameters obtained upon convergence of training are frozen and used as the fingerprint spectrum visual pattern feature extractor for an inference phase.

Specifically, in the step S5, fingerprint spectrum visual pattern features are extracted from the set of protein reference fingerprint spectra of the known bacterial species to obtain a set of protein fingerprint spectrum reference visual pattern features. It should be understood that, in order to construct a high-quality reference benchmark that matches the feature representation of the sample under test, and to overcome the shortcomings of conventional methods that rely on discrete peak information and are susceptible to interference, it is necessary not only to extract robust features from the sample under test, but also to perform the same feature transformation processing on the known bacterial species reference database used for comparison. When the reference database continues to use a traditional spectra or a peak list, it would be impossible to effectively compare and match the visual pattern feature encoding vector of the sample under test, thereby preventing the subsequent query network from functioning. Therefore, in a technical solution of the present disclosure, the fingerprint spectrum visual pattern features are further extracted from the set of protein reference fingerprint spectra of the known bacterial species to obtain a set of protein fingerprint spectrum reference visual pattern feature encoding vectors. That is, raw or preprocessed protein fingerprint spectra of a large number of known bacterial species stored in the bacterial species reference database (including various strains of H. pylori and other related bacterial species thereof) are converted into a standardized database based on visual pattern features using the same feature extraction mechanism as used for the sample under test (for example, using the same dilated convolutional neural network model). Specifically, a corresponding protein fingerprint spectrum reference visual pattern feature encoding vector is generated for each reference bacterial species spectrum, and these vectors are aggregated to form a new reference database based on the feature domain. The purpose of the database is to encode the characteristic identity of each known bacterial species in an accurate and stable manner, ensuring that its representation remains consistent with the feature vectors of the sample under test, thereby laying the foundation for subsequent pattern matching.

Specifically, in one embodiment, the extracting fingerprint spectrum visual pattern features from the set of protein reference fingerprint spectra of the known bacterial species to obtain a set of protein fingerprint spectrum reference visual pattern features includes: processing each protein reference fingerprint spectrum of the known bacterial species in the set of protein reference fingerprint spectra of the known bacterial species through the fingerprint spectrum visual pattern feature extractor based on the dilated convolutional neural network model to obtain a set of protein fingerprint spectrum reference visual pattern feature encoding vectors, which serves as the set of protein fingerprint spectrum reference visual pattern features.

Specifically, in the step S6, a query is performed on the fingerprint spectrum visual pattern features of a protein fingerprint spectrum under test and the set of protein fingerprint spectrum reference visual pattern features to obtain a response feature for the fingerprint spectrum visual pattern feature query. It should be understood that conventional methods based on peak list comparison or overall spectral similarity calculations often remain limited to feature similarity comparison at a superficial level, failing to effectively capture complex pattern variations caused by protein expression differences, sample processing, or instrument fluctuations. As a result, these conventional methods are prone to failure in distinguishing closely related bacterial species or in processing variant spectra. Therefore, merely extracting more robust visual pattern features is insufficient, and an intelligent engine capable of understanding and comparing these complex feature patterns is also required. Accordingly, in a technical solution of the present disclosure, the visual pattern feature encoding vector of a protein fingerprint spectrum under test and the set of protein fingerprint spectrum reference visual pattern feature encoding vectors are further input into a fingerprint spectrum pattern feature query network to obtain a fingerprint spectrum visual pattern feature query-response encoding vector. Particularly, the fingerprint spectrum pattern feature query network is capable of performing advanced and deep-level comparisons between the visual pattern features of the sample under test and numerous reference pattern features in the reference database, ultimately producing a discriminative signal capable of accurately reflecting the identity of the sample under test. The fingerprint spectrum pattern feature query network is capable of performing both individual semantic query and self-attention aggregation functions. First, it can employ an individual semantic query unit to meticulously evaluate the semantic correlation between the feature vector under test and each reference feature vector. “Semantic” stated herein refers to the species-specific biological information contained in the mass spectrometry visual pattern. This is not merely the computation of a simple similarity score, but rather the generation of individual semantic query score encoding vector for the visual pattern of the protein fingerprint spectrum under test that contains multi-dimensional matching information. More importantly, the network then employs a self-attention aggregation strategy and considers the self-distribution characteristics of the feature set, thereby achieving context-aware match evaluation. This means that the network does not treat each test-reference pair match result in isolation, but rather considers the sample under test in the context of the entire reference database, dynamically determining which reference patterns have the most significant and consistent matches with the sample under test on a global scale, and adjusting weights accordingly to effectively aggregate information. The fingerprint spectrum visual pattern feature query-response encoding vector realizes a transition from scattered local feature matching to unified global semantic determination, providing a solid and high-confidence basis for determining the final species detection result, and thereby firmly supporting the objective of the technical solution, namely achieving more reliable and accurate mass spectrometry-based identification of H. pylori.

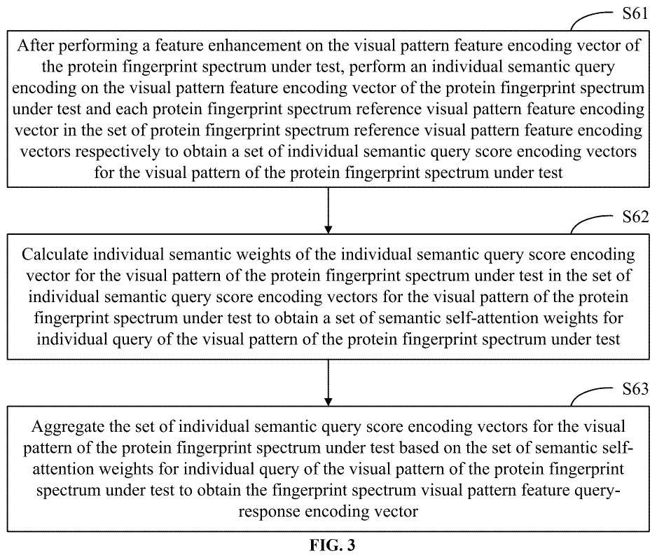

In one embodiment, as shown in FIG. 3, the performing a fingerprint spectrum visual pattern feature query on the protein fingerprint spectrum under test and the set of protein fingerprint spectrum reference visual pattern features to obtain a response feature for the fingerprint spectrum visual pattern feature query includes: S61: after performing a feature enhancement on the visual pattern feature encoding vector of the protein fingerprint spectrum under test, performing an individual semantic query encoding on the visual pattern feature encoding vector of the protein fingerprint spectrum under test and each protein fingerprint spectrum reference visual pattern feature encoding vector in the set of protein fingerprint spectrum reference visual pattern feature encoding vectors respectively to obtain the set of individual semantic query score encoding vectors for the visual pattern of the protein fingerprint spectrum under test; S62. calculating an individual semantic weight of each individual semantic query score encoding vector for the visual pattern of the protein fingerprint spectrum under test in the set of individual semantic query score encoding vectors for the visual pattern of the protein fingerprint spectrum under test to obtain a set of semantic self-attention weights for individual query of the visual pattern of the protein fingerprint spectrum under test; and S63. aggregating the set of individual semantic query score encoding vectors for the visual pattern of the protein fingerprint spectrum under test based on the set of semantic self-attention weights for individual query of the visual pattern of the protein fingerprint spectrum under test to obtain the fingerprint spectrum visual pattern feature query-response encoding vector.

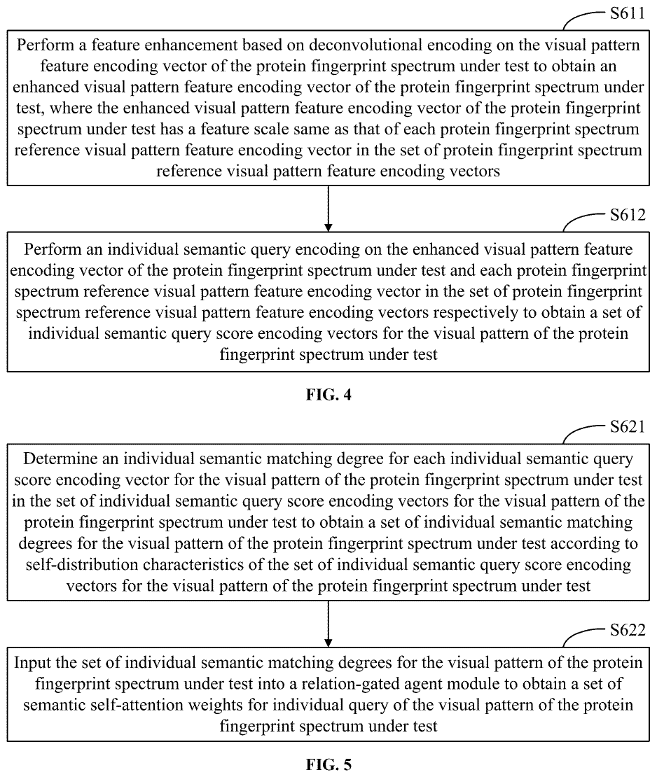

In one embodiment, as shown in FIG. 4, the after performing a feature enhancement on the visual pattern feature encoding vector of the protein fingerprint spectrum under test, performing an individual semantic query encoding on the visual pattern feature encoding vector of the protein fingerprint spectrum under test and each protein fingerprint spectrum reference visual pattern feature encoding vector in the set of protein fingerprint spectrum reference visual pattern feature encoding vectors respectively to obtain a set of individual semantic query score encoding vectors for the visual pattern of the protein fingerprint spectrum under test includes: S611: performing a feature enhancement based on deconvolutional encoding on the visual pattern feature encoding vector of the protein fingerprint spectrum under test to obtain an enhanced visual pattern feature encoding vector of the protein fingerprint spectrum under test, where the enhanced visual pattern feature encoding vector of the protein fingerprint spectrum under test has a feature scale same as that of each protein fingerprint spectrum reference visual pattern feature encoding vector in the set of protein fingerprint spectrum reference visual pattern feature encoding vectors. Specifically, this process can be expressed by the following formula:

$${{V}_{1}}^{′} = \frac{{‖ {V}_{1} ‖}^{2}}{1 + {‖ {V}_{1} ‖}^{2}} \frac{{f}_{deconv} ( {V}_{1} , {W}_{deconv} )}{‖ {V}_{1} ‖} ;$$

where V₁ denotes the visual pattern feature encoding vector of the protein fingerprint spectrum under test, f_{deconv} denotes a deconvolution code, W_{deconv} denotes a deconvolution weight matrix, ∥⋅∥ denotes an L1 norm of a vector, and V₁′ denotes an enhanced visual pattern feature encoding vector of the protein fingerprint spectrum under test.

Specifically, the performing a feature enhancement based on deconvolutional encoding on the visual pattern feature encoding vector of the protein fingerprint spectrum under test has a core effect of significantly improving the expressive capacity and semantic richness of the features. Deconvolutional encoding mechanism (also known as inverse convolution or transposed convolution) learns data-driven features to generate upsampling weights, thereby enabling reconstruction and enhancement of the features in a high-dimensional space while retaining key structural information of the original spectrum. Specifically, this operation not only enhances a spatial resolution of features of the sample under test and strengthens the representation of microscopic patterns and global structures, but also adaptively enhances the content of the original encoding vector through a trainable weight system, thereby effectively recovering, complementing, or highlighting differential and discriminative features in the fingerprint spectrum that may exist latently but are not fully expressed by the shallow encoding. Compared with direct linear transformation or ordinary interpolation, the data-adaptive feature enhancement achieved through deconvolutional encoding significantly expands a basis for comparison between the sample and the database reference feature vectors, ensuring that each vector is mapped into the same higher-level feature space. In addition, the deconvolution process guarantees complete consistency of the enhanced vector with all visual pattern feature encoding vectors of the protein fingerprint spectrum in the reference database in terms of feature scale, dimension, and internal structural distribution. This standardization not only provides a technical guarantee for subsequent fair matching, but also significantly improves the generalization and robustness of the pattern recognition system.

S612. Performing an individual semantic query encoding on the enhanced visual pattern feature encoding vector of the protein fingerprint spectrum under test and each protein fingerprint spectrum reference visual pattern feature encoding vector in the set of protein fingerprint spectrum reference visual pattern feature encoding vectors respectively to obtain the set of individual semantic query score encoding vectors for the visual pattern of the protein fingerprint spectrum under test. Specifically, this process can be expressed by the following formula:

V₂={V₂₁,V₂₂, . . . ,V₂ᵢ. . . ,V₂ₙ};

Rᵢ=tanh{Wᵢ[V₁′;V₂ᵢ]+bᵢ};
where V₂ denotes a set of protein fingerprint spectrum reference visual pattern feature encoding vector; V₂₁, V₂₂, V₂ᵢ, V₂ₙ denote a first, a second, an ith, and an nth protein fingerprint spectrum reference visual pattern feature encoding vectors in the set, respectively; Wᵢ and bᵢ denote a trainable weight matrix and a trainable bias vector, respectively; [⋅;⋅] denotes a vector concatenation operation, tanh(⋅) denotes a hyperbolic tangent function; and Rᵢ denotes an ith individual semantic query score encoding vector for the visual pattern of the protein fingerprint spectrum under test.

Specifically, enhanced visual pattern feature encoding vector of the protein fingerprint spectrum under test and each protein fingerprint spectrum reference visual pattern feature encoding vector in the set of protein fingerprint spectrum reference visual pattern feature encoding vectors are subjected to individual semantic query encoding to obtain a set of individual semantic query score encoding vectors for the visual pattern of the protein fingerprint spectrum under test. An individual semantic query encoding module, through deep neural network-based feature interaction, achieves the practical effect of automatically learning and quantifying the deep semantic correlations and precise matching degree between corresponding feature pairs that consist of enhanced visual pattern feature encoding vector of the protein fingerprint spectrum under test and each protein fingerprint spectrum reference visual pattern feature encoding vector. For example, different H. pylori bacterial species or closely related species under the same disease context may have overall similar protein fingerprint visual patterns, but they exhibit significant differences in the underlying structural distributions, peak region configurations, or sub-block patterns. Individual semantic querying enables high-dimensional encoding of this series of semantic distinctions within the matching vectors. The individual semantic query score encoding vector for the visual pattern of the protein fingerprint spectrum under test is not a single score, but reflects a full range of association patterns of the corresponding feature pairs in a multi-semantic subspace, such that the pairing between the spectrum under test and all reference spectra in the database show a rich and detailed dynamic feature relationship distribution.

In one embodiment, as shown in FIG. 5, the calculating an individual semantic weight of each individual semantic query score encoding vector for the visual pattern of the protein fingerprint spectrum under test in the set of individual semantic query score encoding vectors for the visual pattern of the protein fingerprint spectrum under test to obtain a set of semantic self-attention weights for individual query of the visual pattern of the protein fingerprint spectrum under test respectively includes: S621. determining an individual semantic matching degree for each individual semantic query score encoding vector for the visual pattern of the protein fingerprint spectrum under test in the set of individual semantic query score encoding vectors for the visual pattern of the protein fingerprint spectrum under test to obtain a set of individual semantic matching degrees for the visual pattern of the protein fingerprint spectrum under test according to self-distribution characteristics of the set of individual semantic query score encoding vectors for the visual pattern of the protein fingerprint spectrum under test; and S622. inputting the set of individual semantic matching degrees for the visual pattern of the protein fingerprint spectrum under test into a relation-gated agent module to obtain a set of semantic self-attention weights for individual query of the visual pattern of the protein fingerprint spectrum under test.

In one embodiment, in the step S621, the determining an individual semantic matching degree for each individual semantic query score encoding vector for the visual pattern of the protein fingerprint spectrum under test in the set of individual semantic query score encoding vectors for the visual pattern of the protein fingerprint spectrum under test to obtain a set of individual semantic matching degrees for the visual pattern of the protein fingerprint spectrum under test according to self-distribution characteristics of the set of individual semantic query score encoding vectors for the visual pattern of the protein fingerprint spectrum under test includes: performing intrinsic alignment calibration on each individual semantic query score encoding vector for the visual pattern of the protein fingerprint spectrum under test in the set of individual semantic query score encoding vectors for the visual pattern of the protein fingerprint spectrum under test to obtain a set of calibrated individual semantic query score encoding vectors for the visual pattern of the protein fingerprint spectrum under test. Specifically, this process can be expressed by the following formula:

$${V}_{3 i} = [ {V}_{1}^{′} ; {V}_{2 i} ] ;$$

$${V}_{4 i} = {W}_{4 i} {V}_{3 i} - {V}_{3 i} ;$$

$$g = \frac{// {V}_{4 i} {//}^{2}}{1 + // {V}_{4 i} {//}^{2} ;}$$

$${S}_{i} = {V}_{4 i} / {R}_{i} ;$$

$${R}_{i}^{′} = {R}_{i} + {gS}_{i} {V}_{4 i} ;$$

where V₄ᵢ denotes a query potential vector for the visual pattern of the protein fingerprint spectrum under test; W₄ᵢ denotes a projection weight matrix; V₃ᵢ denotes a query interaction encoding vector for the visual pattern of the protein fingerprint spectrum under test; g denotes a coupling constant, for example, a calculation method same as that for deconvolutional enhancement is adopted to maintain symmetry; Sᵢ denotes a query covariant matrix for the visual pattern of the protein fingerprint spectrum under test; anu

$${R}_{i}^{′}$$

denotes an ith calibrated individual semantic query score encoding vector for the visual pattern of the protein fingerprint spectrum under test.

It should be understood that the individual semantic query encoding is performed by directly concatenating the enhanced visual pattern feature encoding vector of the protein fingerprint spectrum under test with the corresponding reference visual pattern feature encoding vector of the protein fingerprint spectrum in the database, thereby generating the individual semantic query score encoding vectors for the visual pattern of the protein fingerprint spectrum under test. In this process, it is to optimize the representation by leveraging the dynamic characteristics in the mapping from an original feature space to a semantic query encoding space, and focus is placed on calibrating the inherent alignment between the feature space and the semantic query encoding space. This significantly improves the representation accuracy of the individual semantic query score encoding vectors for the visual pattern of the protein fingerprint spectrum under test.

Specifically, the enhanced visual pattern feature encoding vector of the protein fingerprint spectrum under test and each protein fingerprint spectrum reference visual pattern feature encoding vector are integrated through a concatenation operation to form a dynamic eigenvector representation, namely, the query interaction encoding vector for the visual pattern of the protein fingerprint spectrum under test. The query interaction encoding vector for the visual pattern of the protein fingerprint spectrum under test is further subjected to spatial mapping to obtain its projected representation onto an eigenstate of the semantic query encoding, namely, the query potential vector for the visual pattern of the protein fingerprint spectrum under test. On this basis, inherent alignment is applied to the mapped representation of the query potential vector for the visual pattern of the protein fingerprint spectrum under test. The inherent alignment uses the coupling constant setting same as that in the deconvolution-based feature enhancement step, which effectively ensures the symmetry and mathematical consistency of the entire mapping process. In addition, by rationally constructing and solving the covariant matrix of the query covariant matrix for the visual pattern of the protein fingerprint spectrum under test, covariant paths are constrained under canonical symmetric conditions, thereby fixing the mapping canonicalization. This mechanism enhances the representational accuracy of the individual semantic query score encoding vectors for the visual pattern of the protein fingerprint spectrum under test in a high-dimensional semantic space.

According to the distribution characteristics of the set of calibrated individual semantic query score encoding vectors for the visual pattern of the protein fingerprint spectrum under test, an individual semantic matching degree for each calibrated individual semantic query score encoding vector for the visual pattern of the protein fingerprint spectrum under test is calculated to obtain the set of individual semantic matching degrees for the visual pattern of the protein fingerprint spectrum under test. Specifically, this process can be expressed by the following formula:

$${a}_{i} = softmax ( \frac{{\sum }_{k \neq i}^{n} exp ( {R}_{i}^{′} ⊗ {R}_{k}^{′ T} )}{{‖ {R}_{i}^{′} ‖}^{2}} ) ;$$

where n denotes a number of

$${R}_{i}^{′} ; {R}_{k}^{′}$$

denotes an kth calibrated individual semantic query score encoding vector for the visual pattern of the protein fingerprint spectrum under test;

$${R}_{k}^{′ T}$$

denotes a transposed vector of

$${R}_{k}^{′} ;$$

exp denotes a natural exponential function with base e; softmax(⋅) denotes a function of softmax; and aᵢ denotes an individual semantic matching degree for the visual pattern of the protein fingerprint spectrum under test.

Specifically, the calculation of the individual semantic matching degree is not only based on numerical characteristics of each calibrated individual semantic query score encoding vector for the visual pattern of the protein fingerprint spectrum under test, but also based on the distribution characteristics of the set of calibrated individual semantic query score encoding vectors for the visual pattern of the protein fingerprint spectrum under test. In this way, the feature pairs that are capable of significantly distinguishing the spectrum under test from the reference database and that have high biological relevance and discriminative power are assigned higher matching degrees, while noisy and redundant feature pairs that have weak discriminative power are automatically down-weighted. Ultimately, all individual semantic matching degrees jointly form the set of individual semantic matching degrees for the visual pattern of the protein fingerprint spectrum under test.

Specifically, a calculation process of inputting the set of individual semantic matching degrees for the visual pattern of the protein fingerprint spectrum under test into a relation-gated agent module to obtain a set of semantic self-attention weights for individual query of the visual pattern of the protein fingerprint spectrum under test is expressed by the following formula:

$${w}_{i} = mask ( {a}_{i} ) = \{ \begin{matrix}{a}_{i} & {a}_{i} \geq τ \\ 0 & {a}_{i} < τ\end{matrix} ;$$

where mask denotes a masking function, τ denotes a preset threshold (such as 0.5, this is merely an illustrative example and may be adjusted according to actual situation), and wᵢ denotes a set of semantic self-attention weights for individual query of the visual pattern of the protein fingerprint spectrum under test.

Specifically, the relation-gated agent module can, through a trainable gating mechanism, effectively retain the individual semantic matching degrees for the visual pattern of the protein fingerprint spectrum under test that are most relevant to discriminating the target bacterial species such as H. pylori, and can automatically suppress the spurious high scores, weak correlations, or redundant pairings that may adversely affect the overall decision outcome at the same time. Based on contextual understanding of the global feature space, the relation-gated agent module achieves dynamic weight assignment for the individual semantic matching degrees for the visual pattern of the protein fingerprint spectrum under test in subsequent self-attention aggregation, ensuring that the final focus of the self-attention mechanism is highly consistent with the actual discrimination requirements.

Specifically, a calculation process of aggregating the set of individual semantic query score encoding vectors for the visual pattern of the protein fingerprint spectrum under test based on the set of semantic self-attention weights for individual query of the visual pattern of the protein fingerprint spectrum under test to obtain the fingerprint spectrum visual pattern feature query-response encoding vector is expressed by the following formula:

vₚ=ΣᵢwᵢRᵢ;
where vₚ denotes a fingerprint spectrum visual pattern feature query-response encoding vector.

By performing deep modeling in the feature domain, an individual semantic query score encoding vector between each visual pattern of the protein fingerprint spectrum under test and the database reference pattern is obtained, and each encoding vector is assigned an adaptive semantic self-attention weight for individual query by leveraging the contextual distribution characteristics of the feature set and the relation-gated agent mechanism. These weights reflect the actual contribution and discriminative power of each encoding vector in the overall feature representation, effectively highlighting highly discriminative matching information while attenuating or suppressing noise and redundancy. On this basis, the aggregation step dynamically performs a linear combination of all individual semantic query score encoding vectors using the self-attention weights as a weighting factor. In the aggregation process, not only is local matching information at a microscopic level integrated according to its importance, but also a global semantic abstraction and consistent representation of the overall protein fingerprint visual structure is achieved.

Specifically, in the step S7, determining a bacterial species detection result based on the response feature for the fingerprint spectrum visual pattern feature query. In one embodiment, the determining a bacterial species detection result based on the response feature for the fingerprint spectrum visual pattern feature query includes: processing the fingerprint spectrum visual pattern feature query-response encoding vector through a classifier-based bacterial species detector to obtain the bacterial species detection result, where the bacterial species detection result is used to represent a bacterial species category label. Specifically, a Softmax classifier is widely used in the final multi-class discrimination module after deep learning features due to its rigorous structure, strong differentiability, and suitability for multi-class probabilistic modeling. In one specific embodiment of the present disclosure, the fingerprint spectrum visual pattern feature query-response encoding vector is input into the Softmax classifier. When the fingerprint spectrum visual pattern feature query-response encoding vector is a fixed-length high-dimensional vector, and the system reference database contains m bacterial species categories, for each category i, the Softmax classifier first multiplies by a learnable weight vector corresponding to the category and adds the corresponding bias to obtain a score. Exponential calculation is then performed to generate a probability distribution, and a category corresponding to a maximum pi is output as the bacterial species detection result. The probability distribution can also be directly interpreted as a confidence indicator of the model.

In summary, the Helicobacter pylori detection method based on mass spectrometry identification provided in the present disclosure first acquires MALDI mass spectra of the sample under test to obtain its protein fingerprint spectrum. The spectrum is then preprocessed to remove noise and is standardized, followed by extraction of visual pattern features of the feature under test with discriminative ability. Reference visual pattern features of the known bacterial species are extracted from a previously constructed reference database. Finally, a complex matching relationship between the features under test and the reference feature is identified by introducing protein fingerprint spectrum pattern feature query-response analysis based on a feature domain to obtain a query-response encoding representation of the fingerprint spectrum visual pattern feature, and a final bacterial species detection result is determined based on the query response. This strategy of feature extraction and query network matching based on the visual pattern features is intended to fully exploit deep pattern information embedded in mass spectrometry data, thereby enabling more reliable mass spectrometry identification of H. pylori.

The fundamental principles of the present disclosure have been described above in combination with specific embodiments. However, it should be noted that the advantages, benefits, and effects mentioned in the present disclosure are merely illustrative and not restrictive; they should not be construed as features that must be possessed by each embodiment of the present disclosure. In addition, the specific details of the above embodiments are provided solely for illustrative purposes and to facilitate understanding, and are not intended to limit the present disclosure. These details do not necessarily limit the present disclosure to be implemented using the specific details.

In the above embodiments, the descriptions of all embodiments have their respective focuses. Any part not recorded or described in one embodiment may be referred to in the relevant descriptions of other embodiments. In several embodiments provided in the present disclosure, it should be understood that the disclosed device and method can be implemented in other ways. For example, the device embodiments described above are merely illustrative, the division of the modules is only a logical function division, and there may be additional divisions in actual implementation. The modules described as separate components may or may not be physically separated, and a component displayed as a module may or may not be a physical unit, that is, the component may be located at one place, or distributed on a plurality of network units. Part or all of the modules may be selected according to actual requirements to achieve the objective of the solution of the embodiments.

In addition, the functional modules in each embodiment of the present disclosure may be integrated into one processing unit, may exist separately as individual physical units, or two or more units may be integrated into one unit. The integrated unit can be implemented either in the form of hardware or in a combination of hardware and software functional modules.

Finally, it should be noted that the above description has been provided for purposes of illustration and description. Although the present disclosure is described in detail with reference to the above embodiments, persons of ordinary skill in the art should understand that they may still make modifications or equivalent replacements to the technical solutions of the present disclosure without departing from the spirit and scope of the technical solutions of the present disclosure.

What is claimed is:

1. A Helicobacter pylori detection method based on mass spectrometry identification, comprising:
mixing a sample under test with a matrix-assisted laser desorption/ionization (MALDI) matrix to obtain a mixture, spotting the mixture onto a MALDI target plate, and acquiring a protein fingerprint spectrum using a matrix-assisted laser desorption/ionization time-of-flight (MALDI-TOF) mass spectrometer;
extracting a set of protein reference fingerprint spectra of a known bacterial species from a bacterial species reference database;
preprocessing the protein fingerprint spectrum to obtain a preprocessed protein fingerprint spectrum;
extracting a fingerprint spectrum visual pattern feature from the preprocessed protein fingerprint spectrum to obtain a visual pattern feature of a protein fingerprint spectrum under test, wherein the step comprises: processing the preprocessed protein fingerprint spectrum through a fingerprint spectrum visual pattern feature extractor based on a dilated convolutional neural network model to obtain a visual pattern feature encoding vector of the protein fingerprint spectrum under test, wherein the visual pattern feature encoding vector of the protein fingerprint spectrum under test serves as the visual pattern feature of the protein fingerprint spectrum under test;
extracting fingerprint spectrum visual pattern features from the set of protein reference fingerprint spectra of the known bacterial species to obtain a set of protein fingerprint spectrum reference visual pattern features, wherein the step comprises: processing each protein reference fingerprint spectrum of the known bacterial species in the set of protein reference fingerprint spectra of the known bacterial species through the fingerprint spectrum visual pattern feature extractor based on the dilated convolutional neural network model to obtain a set of protein fingerprint spectrum reference visual pattern feature encoding vectors, wherein the set of protein fingerprint spectrum reference visual pattern feature encoding vectors serves as the set of protein fingerprint spectrum reference visual pattern features;
performing a fingerprint spectrum visual pattern feature query on the protein fingerprint spectrum under test and the set of protein fingerprint spectrum reference visual pattern features to obtain a response feature for the fingerprint spectrum visual pattern feature query; and
determining a bacterial species detection result based on the response feature for the fingerprint spectrum visual pattern feature query; wherein
the performing the fingerprint spectrum visual pattern feature query on the protein fingerprint spectrum under test and the set of protein fingerprint spectrum reference visual pattern features to obtain the response feature for the fingerprint spectrum visual pattern feature query comprises:
after performing a feature enhancement on the visual pattern feature encoding vector of the protein fingerprint spectrum under test to obtain an enhanced visual pattern feature encoding vector of the protein fingerprint spectrum under test, performing an individual semantic query encoding on the enhanced visual pattern feature encoding vector of the protein fingerprint spectrum under test and each protein fingerprint spectrum reference visual pattern feature encoding vector in the set of protein fingerprint spectrum reference visual pattern feature encoding vectors respectively to obtain a set of individual semantic query score encoding vectors for a visual pattern of the protein fingerprint spectrum under test;
calculating an individual semantic weight of each individual semantic query score encoding vector for the visual pattern of the protein fingerprint spectrum under test in the set of individual semantic query score encoding vectors for the visual pattern of the protein fingerprint spectrum under test to obtain a set of semantic self-attention weights for individual query of the visual pattern of the protein fingerprint spectrum under test; and
aggregating the set of individual semantic query score encoding vectors for the visual pattern of the protein fingerprint spectrum under test based on the set of semantic self-attention weights for individual query of the visual pattern of the protein fingerprint spectrum under test to obtain a fingerprint spectrum visual pattern feature query-response encoding vector.

2. The Helicobacter pylori detection method based on the mass spectrometry identification according to claim 1, wherein the preprocessing comprises baseline correction, noise smoothing, peak detection, and alignment.

3. The Helicobacter pylori detection method based on the mass spectrometry identification according to claim 2, wherein the after performing the feature enhancement on the visual pattern feature encoding vector of the protein fingerprint spectrum under test to obtain the enhanced visual pattern feature encoding vector of the protein fingerprint spectrum under test, performing the individual semantic query encoding on the enhanced visual pattern feature encoding vector of the protein fingerprint spectrum under test and each protein fingerprint spectrum reference visual pattern feature encoding vector in the set of protein fingerprint spectrum reference visual pattern feature encoding vectors respectively to obtain the set of individual semantic query score encoding vectors for the visual pattern of the protein fingerprint spectrum under test comprises:
performing the feature enhancement based on deconvolutional encoding on the visual pattern feature encoding vector of the protein fingerprint spectrum under test to obtain the enhanced visual pattern feature encoding vector of the protein fingerprint spectrum under test, wherein the enhanced visual pattern feature encoding vector of the protein fingerprint spectrum under test has a feature scale same as a feature scale of each protein fingerprint spectrum reference visual pattern feature encoding vector in the set of protein fingerprint spectrum reference visual pattern feature encoding vectors; and
performing the individual semantic query encoding on the enhanced visual pattern feature encoding vector of the protein fingerprint spectrum under test and each protein fingerprint spectrum reference visual pattern feature encoding vector in the set of protein fingerprint spectrum reference visual pattern feature encoding vectors respectively to obtain the set of individual semantic query score encoding vectors for the visual pattern of the protein fingerprint spectrum under test.

4. The Helicobacter pylori detection method based on the mass spectrometry identification according to claim 3, wherein the calculating the individual semantic weight of each individual semantic query score encoding vector for the visual pattern of the protein fingerprint spectrum under test in the set of individual semantic query score encoding vectors for the visual pattern of the protein fingerprint spectrum under test to obtain the set of semantic self-attention weights for individual query of the visual pattern of the protein fingerprint spectrum under test comprises:
determining an individual semantic matching degree for each individual semantic query score encoding vector for the visual pattern of the protein fingerprint spectrum under test in the set of individual semantic query score encoding vectors for the visual pattern of the protein fingerprint spectrum under test to obtain a set of individual semantic matching degrees for the visual pattern of the protein fingerprint spectrum under test according to self-distribution characteristics of the set of individual semantic query score encoding vectors for the visual pattern of the protein fingerprint spectrum under test; and
inputting the set of individual semantic matching degrees for the visual pattern of the protein fingerprint spectrum under test into a relation-gated agent module to obtain the set of semantic self-attention weights for individual query of the visual pattern of the protein fingerprint spectrum under test.

5. The Helicobacter pylori detection method based on the mass spectrometry identification according to claim 4, wherein the determining the individual semantic matching degree for each individual semantic query score encoding vector for the visual pattern of the protein fingerprint spectrum under test in the set of individual semantic query score encoding vectors for the visual pattern of the protein fingerprint spectrum under test to obtain the set of individual semantic matching degrees for the visual pattern of the protein fingerprint spectrum under test according to the self-distribution characteristics of the set of individual semantic query score encoding vectors for the visual pattern of the protein fingerprint spectrum under test comprises:
performing intrinsic alignment calibration on each individual semantic query score encoding vector for the visual pattern of the protein fingerprint spectrum under test in the set of individual semantic query score encoding vectors for the visual pattern of the protein fingerprint spectrum under test to obtain a set of calibrated individual semantic query score encoding vectors for the visual pattern of the protein fingerprint spectrum under test; and
calculating an individual semantic matching degree for each calibrated individual semantic query score encoding vector for the visual pattern of the protein fingerprint spectrum under test respectively according to self-distribution characteristics of the set of calibrated individual semantic query score encoding vectors for the visual pattern of the protein fingerprint spectrum under test to obtain the set of individual semantic matching degrees for the visual pattern of the protein fingerprint spectrum under test.

6. The Helicobacter pylori detection method based on the mass spectrometry identification according to claim 5, wherein the determining the bacterial species detection result based on the response feature for the fingerprint spectrum visual pattern feature query comprises: processing the fingerprint spectrum visual pattern feature query-response encoding vector through a classifier-based bacterial species detector to obtain the bacterial species detection result, wherein the bacterial species detection result is configured to represent a bacterial species category label.
